(19) United States
(12) Patent Application Publication
(10) Pub. No.: US 2023/0310007 A1
(43) Pub. Date: Oct. 5, 2023

(54) MULTICLIP ADAPTOR FOR SURGICAL CLIP DRIVERS

(71) Applicant: University of Saskatchewan, Saskatoon (CA)

(72) Inventors: Yigang Luo, Windsor (CA); Wen Jun Zhang, Saskatoon (CA); Shaoqing Du, Saskatoon (CA); Robert Bigsby, Saskatoon (CA); Stephanie Ruscheinsky, Saskatoon (CA); Chenwang Yuan, Saskatoon (CA)

(21) Appl. No.: 18/023,429

(22) PCT Filed: Aug. 31, 2021
(86) PCT No.: PCT/CA2021/051202

§ 371 Date: Feb. 27, 2023

Related U.S. Application Data
(60) Provisional application No. 63/072,524, filed on Aug. 31, 2020.

(51) Int. Cl. A61B 17/122

(52) U.S. Cl. CPC A61B 17/1222

(57) ABSTRACT

An adaptor for a surgical clip driver is disclosed herein. The adaptor includes a first body and a second body. Each of the first body and the second body has an opening extending inwardly that is configured to receive a portion of a jaw of the surgical clip driver. The first body also includes a first body retaining member configured to retain a first portion of each of the two or more surgical clips and the second body also includes a second body retaining member configured to retain a second portion of each of the two or more surgical clips. When the first body and the second body are received on the surgical clip driver, the first body retaining member and the second body retaining member face each other and are aligned with each other to receive and retain the two or more surgical clips. A cartridge for retaining surgical clips is also disclosed herein.

### CROSS REFERENCE

This application claims the benefit of U.S. Provisional Patent Application No. 63/072,524 filed on Aug. 31, 2021 and the entire contents of U.S. Provisional Patent Application No. 63/072,524 are hereby incorporated herein in their entirety.

### TECHNICAL FIELD

This disclosure relates generally to surgical clip drivers, and more specifically, to adaptors for simultaneously applying more than one surgical clip using a surgical clip driver.

### BACKGROUND

In surgical operations, it is often necessary to apply hemostatic clips to blood vessels to restrict the movement of blood. Hemostatic clips are commonly used in both open surgeries, where anatomic cavities are cut open with large incisions so that the surgeon has a full view of the structures and organs involved in the surgery (e.g. without the assistance of a camera), and minimally invasive surgeries, such as but not limited to laparoscopic surgeries and thoracoscopic surgeries, where incisions in the patient are much smaller and the surgeon does not have a full view of the structures and organs involved.

Minimally invasive surgeries are becoming increasingly common procedures due to their advantages of smaller incisions, less trauma, reduced pain, shorter hospital stays, and quicker recovery when compared to open surgery.

Despite these advantages, minimally invasive surgeries commonly take longer operating time. Studies have shown that about 30% of laparoscopic surgical procedure time is spent on instrument exchange, including multiple repetitions of reloading a surgical ligation single-clip conventional driver with new clips.

Further, instrument exchange during minimally invasive surgeries may result in traumatic complications. The use of conventional clip drivers that place one clip individually at a time may therefore increase the risk of surgical complications due to trauma from repetitive instrument exchange and misplacement of the clip driver in the surgical field.

Typically, applying multiple surgical ligation clips prior to transection is safer and more common practice than applying a single clip. Two to three surgical ligation clips are typically used at each transection site, such as but not limited to a cystic duct or a small vessel. However, applying two or three surgical ligation clips can be time consuming as conventional clip drivers fire clips individually. In addition, applying multiple surgical ligation clips with conventional clip drivers often results in inaccurate placement anatomically.

Accordingly, there is a need for a new device for the accurate, simultaneous placement of more than one surgical ligation clip during a surgery.

### SUMMARY

In accordance with a broad aspect, there is provided an adaptor for a surgical clip driver for applying two or more surgical clips. The adaptor includes a first body having a first end, a second end, an inner surface extending between the first end and the second end and an outer surface opposed to the inner surface and extending between the first end and the second end. The first end has an opening extending inwardly into the first body, the opening being configured to receive a portion of a first jaw of the surgical clip driver. The first body also includes a first body retaining member configured to retain a first portion of each of the two or more surgical clips. The adaptor also includes a second body having a first end, a second end, an inner surface extending between the first end and the second end and an outer surface opposed to the inner surface and extending between the first end and the second end. The first end of the second body has an opening extending inwardly into the second body, the opening being configured to receive a portion of a second jaw of the surgical clip driver. The second body also includes a second body retaining member configured to retain a second portion of each of the two or more surgical clips. When the first body and the second body are received on the surgical clip driver, the first body retaining member and the second body retaining member face each other and are aligned with each other to receive and retain each of the two or more surgical clips.

In at least one embodiment, the first body retaining member is integral with the first body and includes two or more slots extending inwardly into the first body and the second body retaining member is integral with the second body and includes two or more slots extending inwardly into the second body.

In at least one embodiment, the two or more slots of the first body are parallel with each other and extend between the first end of the first body and the second end of the first body and the two or more slots of the second body are parallel with each other and extend between the first end of the second body and the second end of the second body.

In at least one embodiment, the two or more slots of the first body include two pairs of slots and the two or more slots of the second body include two pairs of slots.

In at least one embodiment, each the two pairs of slots are spaced apart from each other by a distance that is greater than a distance between the slots of each pair of slots.

In at least one embodiment, the two or more slots of the first body include three slots.

In at least one embodiment, the three slots include one pair of slots spaced apart from a single slot.

In at least one embodiment, the pair of slots is spaced apart from the single slot by a distance that is greater than a distance between the slots of the pair of slots.

In at least one embodiment, each slot has an inner surface shaped to correspond with an outer surface of a surgical clip such that upon applying a force on the surgical clip in a direction towards the inner surface of the slot, the surgical clip is frictionally retained in the slot.

In at least one embodiment, the first body retaining member includes at least one first slot member coupled to the inner surface of the first body, the first slot member having a slot extending inwardly into a body of the first slot member.

In at least one embodiment, the first body retaining member includes three first slot members.

In at least one embodiment, the three first slot members include one pair of first slot members spaced apart from a single first slot member.

In at least one embodiment, the pair of first slot members is spaced apart from the single first slot member by a distance that is greater than a distance between the slots of the pair of first slot members.

In at least one embodiment, the second body retaining member includes at least one second slot member coupled to the inner surface of the second body, the second slot member having a slot extending inwardly into a body of the second slot member.

In at least one embodiment, the second body retaining member includes three second slot members.

In at least one embodiment, the three second slot members include one pair of second slot members spaced apart from a single second slot member.

In at least one embodiment, the second body retaining member includes at least one second slot member coupled to the inner surface of the second body, the second slot member having a slot extending inwardly into a body of the second slot member.

In at least one embodiment, the pair of second slot members is spaced apart from the single second slot member by a distance that is greater than a distance between the slots of the pair of second slot members.

In accordance with a broad aspect, a method of applying two or more surgical clips using an adaptor described herein is described herein. The method includes placing at least a portion of a first jaw of the surgical clip driver into an opening of a first body of the adaptor; placing at least a portion of a second jaw of the surgical clip driver into an opening of a second body of the adaptor; loading at least two or more surgical clips into retaining members of the first body and the second body; positioning the first body and second body of the adaptor around a selected target clipping tissue; and actuating the surgical clip driver to simultaneously apply the two or more clips to the selected target clipping tissue.

In accordance with a broad aspect, a cartridge for supporting two or more surgical clips is described herein. The cartridge includes a clip support configured to support the two or more surgical clips. The clip support includes a hedge extending upwardly from a base to support the clips in an upside down orientation such that a rear portion of each clip is upwardly exposed. The hedge includes a separator positioned on either side of each clip to stabilize the clips from each other when the clip support is loaded with two or more clips. The cartridge also includes a cover with an opening sized and shaped to provide for a clip driver to simultaneously retrieve the two or more clips from the clip support through the opening.

In at least one embodiment, each of the separators is a flange extending outwardly from an outer surface of the hedge of the clip support.

In at least one embodiment, each of the separators of the clip support are spaced apart from each other by a same distance as a distance between the slots of the adaptor.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a perspective view of a simultaneous multiclip firing adapter unit for a surgical ligation clip driver, according to at least one embodiment.

FIGS. 2A to 2C are top, side and end views, respectively, of the simultaneous multiclip firing adapter unit for a surgical ligation clip driver of FIG. 1.

FIG. 3A is an image of a simultaneous multiclip firing adapter unit according to at least one embodiment inserted on a surgical ligation clip driver.

FIGS. 3B to 3C are views of a simultaneous multiclip firing adapter unit according to at least one embodiment inserted on a surgical ligation clip driver having multiple clips loaded in the adapter unit.

FIG. 4A is an image of four clips that were fired using a simultaneous multiclip firing adapter unit for surgical ligation clip drivers according to at least one embodiment described herein.

FIG. 4B is an image of four clips that were individually placed using a conventional single clip driver.

FIG. 4C is an image of three clips that were individually placed using a conventional single clip driver.

FIG. 5 is a block diagram of a method of simultaneously applying two or more surgical ligation clips.

FIG. 6 is a top view of a conventional cartridge for retaining surgical clips.

FIG. 7A is a front perspective view of a cartridge for retaining surgical clips with a portion of the cover removed, according to at least one embodiment.

FIG. 7B is a rear perspective view of the cartridge of FIG. 7A.

Further aspects and features of the example embodiments described herein will appear from the following description taken together with the accompanying drawings.

### DETAILED DESCRIPTION

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter is not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive-or. That is, “X and/or Y” is intended to mean X, Y or X and Y, for example. As a further example, “X, Y, and/or Z” is intended to mean X or Y or Z or any combination thereof. Also, the expression of A, B and C means various combinations including A; B; C; A and B; A and C; B and C; or A, B and C.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in developing new tools for simultaneously applying more than one surgical clip to a selected target during a surgery.

Adaptors for a surgical clip driver are described herein. The adaptors described herein are appropriate for applying two or more surgical clips to a selected target (e.g. tissue) simultaneously during a surgery. The surgery may be an open surgery or may be a minimally invasive surgery. The adaptors described herein include a pair of bodies (or devices), e.g. made of bronze, plastic or any other appropriate material, for use inside of a human body, that are removably attachable to the tips of a surgical ligation clip driver. Each of the pair of bodies has an external surface (e.g. shaped like a hemi-oval) and an internal (or medial) surface having more than one slot (or holding groove) that is sized and shaped to match the size of and retain a typical surgical clip. The slots, or medial grooves, of each of the pair of bodies are counterpart precise to each other so the slots of the two bodies can act together to act as clip holding sites.

In at least one embodiment, when the pair of bodies (together, the adaptor) are placed on or applied to a surgical ligation clip driver and the user closes clip driver, the internal surface of each slot of each body of the adaptor closes inwardly onto a clip or clips loaded into or retained in the slots and bends each clip around a clip target (e.g. blood vessel). When the clip driver opens again, the clips are simultaneously fired and remain on the clip target.

The adaptors described herein may be adaptable to be placed on the tips of many different clip drivers and may allow for shortened surgical procedure times and reduced risk of surgical complications by reducing the need for users (e.g. surgeons) to repeatedly remove and reload the clip driver. Such improvements could lead to reduced surgical time, lower costs per surgery, better patient care, and better overall outcomes for patients.

It should be understood that although the adaptors described herein are described in the context of being used with a laparoscopic clip driver, as noted above, the adaptors described herein may also be suitable for clip drivers to be used during other types of surgeries, such as but not limited to other types of minimally invasive surgeries (e.g. thoracoscopic surgery, minimally invasive urologic and gynecologic procedures, etc.) as well as open surgeries.

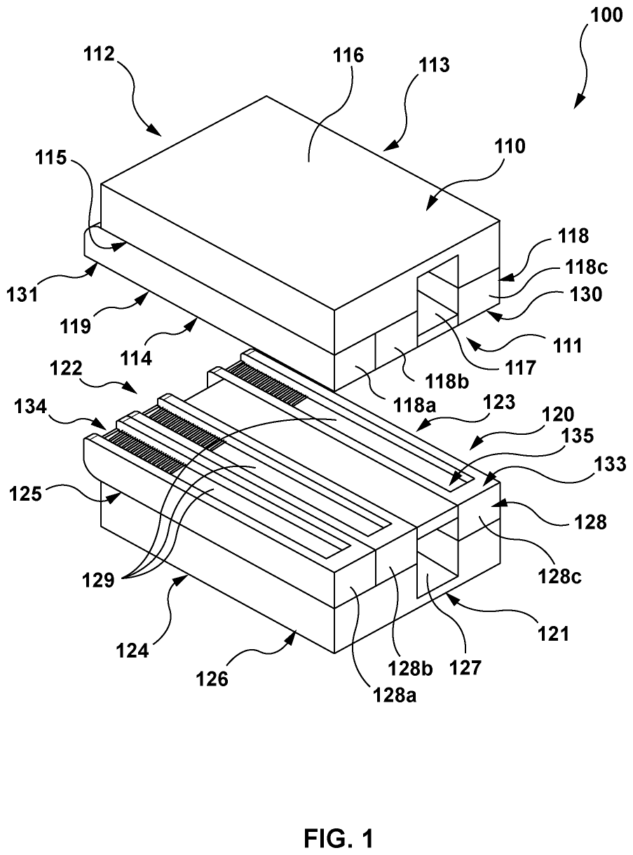

Referring now to FIG. 1, illustrated there is an adaptor 100 for a surgical clip driver. Adaptor 100 includes a first body 110 and a second body 120. First body 110 and second body 120 are opposed to each other and can each receive at least a portion of a surgical clip driver.

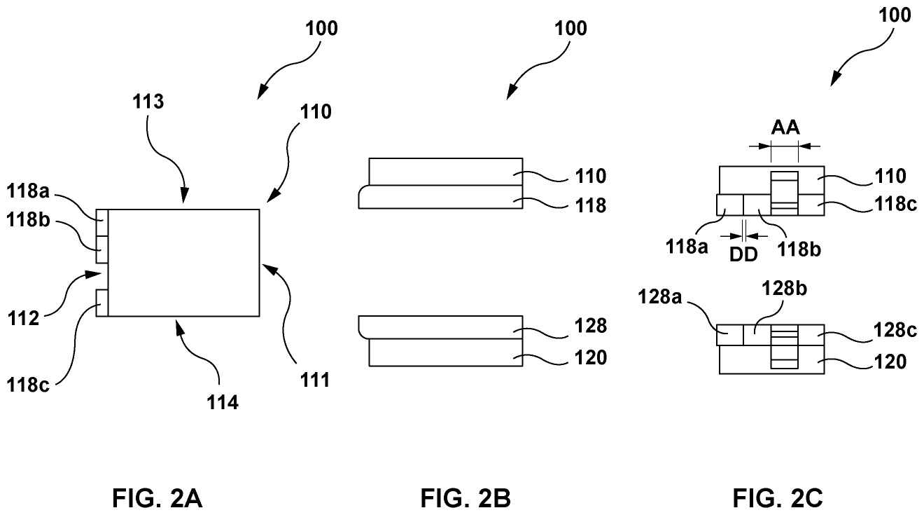

First body 110 has a first end 111, a second end 112, a first side 113 and a second side 114 opposed to first side 113. First body 110 also includes an inner surface 115 extending between the first end 111 and the second end 112 and an outer surface 116 opposed to the inner surface 115 and extending between the first end 111 and the second end 112. In the example embodiment shown in FIGS. 1-2C, the first body 110 has a generally rectangular shape with the first end 111 and the second end 112 being generally planar and parallel to each other and with the first side 113 and the second side 114 being generally planar and parallel to each other. In this embodiment, inner surface 115 and the outer surface 116 are also generally planar surfaces that are parallel to each other, giving the first body 110 a generally cuboid shape.

First end 111 of first body 110 has an opening 117 extending inwardly into the first body 110 towards the second end 112. Opening 117 is configured to receive a portion of a surgical clip driver (e.g. a portion of a first jaw of a surgical clip driver). Opening 117 extends inwardly by a distance that is sufficient to receive a portion of a first jaw of a surgical clip driver, for example, and/or is sized and shaped such that the first jaw of a surgical clip driver can be retained in the first opening 117, such as but not limited to by friction. In at least one embodiment, opening 117 passes completely through the first body 110.

First body 110 also includes a first retaining member 118. In the embodiment shown in FIGS. 1-2C, the first retaining member 118 includes at least one first slot member 118i coupled to the inner surface 115 of the first body 110. Each slot member 118i has a slot 119i extending inwardly into a body of the first slot member 118i. For instance, in the embodiments shown in FIGS. 1-2C, first retaining member 118 includes three first slot members 118a, 118b, 118c that are positioned on the inner surface 115 of the first body 110. Each first slot member 118i is configured to retain at least a portion of one surgical clip.

First retaining member 118 may include a physical mechanism for retaining each of the two or more surgical clips. For example, as shown in the embodiments of FIGS. 1-2C and 3A-3C, first retaining member 118 may include more than one slot member 118i, each slot member 118i having a respective slot 119 therein extending laterally inwardly from second end 112 towards first end 111 and vertically inwardly from an outer surface 130 of the first retaining member 118 towards inner surface 115 of first body 110. Each slot 119 of each slot member 118i is shaped to receive and retain at least a portion of a surgical ligation clip (e.g. by friction). Each slot 119 of each slot member 118i may have an open end 131 and a closed end 132. Open end 131 may be sized and shaped to provide for the surgical clip to be received therein and/or extend therethrough (e.g. extend outwardly from the slot retaining member 118i) once the surgical clip is retained in the slot 119. Open end 131 may also contribute to retaining the surgical clip in the slot 119. Collectively, the slots 119 of the slot members 118i may be parallel with each other and may extend laterally between the first end 111 and the second end 112 of the first body 110 (e.g. from first end 111 to second end 112).

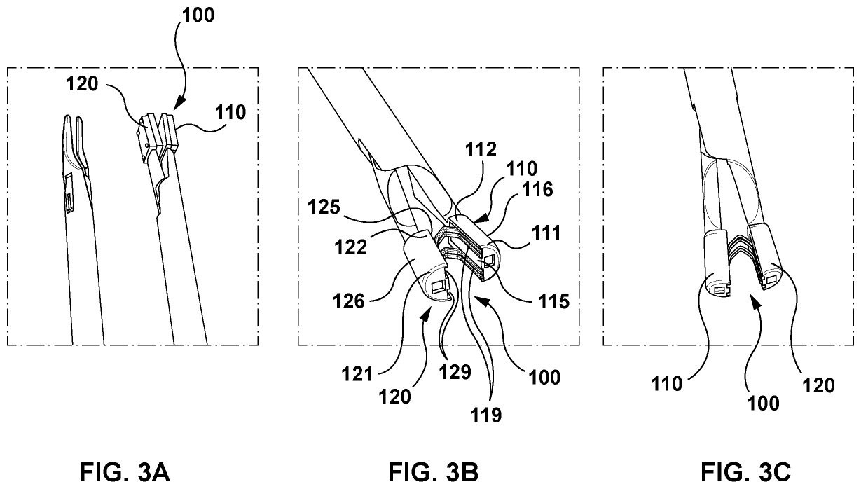

In the embodiments shown in FIGS. 3B-3C, first retaining member 118 is integral with the first body 110 and comprises two or more slots 119. Slots 119 may extend laterally inwardly from second end 112 towards first end 111 and vertically inwardly into the first body 110 from inner surface 115 towards outer surface 116.

It should be understood that the first body 110 may have different shapes beyond those shown in the embodiments provided herein, provided that the first opening 117 is generally positioned on an end of the first body 110 to receive at least a portion of a jaw of a surgical clip driver.

Second body 120 has a first end 121, a second end 122, a first side 123 and a second side 124 opposed to the first side 123. Second body 120 also includes an inner surface 125 extending between the first end 121 and the second end 122 and an outer surface 126 opposed to the inner surface 125 and extending between the first end 121 and the second end 122. In the example embodiment shown in FIGS. 1-2C, the second body 120 has a generally rectangular shape with the first end 121 and the second end 122 being generally planar and parallel to each other and with the first side 123 and the second side 124 being generally planar and parallel to each other. In this embodiment, inner surface 125 and the outer surface 126 are also generally planar surfaces that are parallel to each other, giving the second body 120 a generally cubic shape.

First end 121 of second body 120 has an opening 127 extending inwardly into the second body 120 towards the second end 122. Opening 127 is configured to receive a portion of a surgical clip driver (e.g. a portion of a second jaw of the surgical clip driver). Opening 127 extends inwardly by a distance that is sufficient to receive a portion of a second jaw of a surgical clip driver, for example, and/or is sized and shaped such that the second jaw of a surgical clip driver can be retained in the second opening 127 such as but not limited to by friction. In at least one embodiment, opening 127 passes completely through the second body 120.

Second retaining member 128 may include a physical mechanism for retaining each of the two or more surgical clips. For example, as shown in the embodiments of FIGS. 1-2C and 3A-3C, second retaining member 128 may include more than one slot member 128i, each slot member 128i having a respective slot 129 therein extending laterally inwardly from second end 122 towards first end 121 of second body 120 and vertically inwardly from an outer surface 133 of the first retaining member 128 towards inner surface 125 of second body 120. Each slot 129 of each slot member 128i is shaped to receive and retain at least a portion of a surgical ligation clip (e.g. by friction). Each slot 129 of each slot member 128i may have an open end 134 and a closed end 135. Open end 134 may be sized and shaped to provide for the surgical clip to be received therein and/or extend therethrough (e.g. extend outwardly from the slot retaining member 118i) once the surgical clip is retained in the slot 129. Open end 134 may also contribute to retaining the surgical clip in the slot 129. Collectively, the slots 129 of the slot members 128i may be parallel with each other and may extend laterally between the first end 121 and the second end 122 of the second body 120 (e.g. from first end 121 to second end 122).

In the embodiments shown in FIGS. 3A-3C, second retaining member 128 is integral with the second body 120 and comprises two or more slots 129. Slots 129 may extend laterally inwardly from second end 122 towards first end 121 and vertically inwardly into the second body 120 from inner surface 125 towards outer surface 126.

It should be understood that the second body 120 may have different shapes beyond those shown in the embodiments provided herein, provided that the second opening 127 is generally positioned on an end of the second body 120 to receive at least a portion of a jaw of a surgical clip driver.

When first body 110 and second body 120 are received on a surgical clip driver, first retaining member 118 and the second retaining member 128 may face each other and be aligned with each other to receive and retain each of the two or more surgical clips. FIG. 1 shows first retaining member 118 and the second retaining member 128 each being able to retain three surgical clips.

First retaining member 118 and the second retaining member 128 may include a physical mechanism for retaining each of the two or more surgical clips. For example, as shown in the embodiments shown in both FIGS. 1-2C and 3A-3C, first retaining member 118 may include two or more slots 119 extending laterally inwardly from second end 112 and second retaining member 128 may include two or more slots 129 extending laterally inwardly from the second end 122. As shown in FIGS. 3B and 3C, the slots 119 and 129 of the first retaining member 118 and the second retaining member 128 are aligned such that each slot 119 has a corresponding slot 129 that is aligned therewith to retain a surgical clip.

In at least one embodiment, the slots 119 and/or the slots 129 may be grouped together. As noted above, placing two or more clips immediately adjacent to one another may offer vasculature blocking benefits. For instance, the two or more slots 119 may include two pairs of slots and the two or more slots 129 may include two pairs of slots. In at least one embodiment, each of the two pairs of slots may be spaced apart from each other by a distance AA that is greater than a distance DD between the slots of each pair of slots. An example of this can be seen in FIG. 2C (albeit with a single pair of slots being spaced apart from a single slot).

In at least one embodiment, the two or more slots 119 of the first retaining member 118 are three slots 119a, 119b and 119c. In at least one embodiment, the three slots 119a, 119b and 119c are arranged to include one pair of slots spaced apart from a single slot. Similarly, in this embodiment, in this embodiment, the two or more slots 129 of the second retaining member 128 are three slots 129a, 129b and 129c. In at least one embodiment, the three slots 129a, 129b and 129c are arranged to include one pair of slots spaced apart from a single slot, the one pair of slots being opposed to the pair of slots of the first retaining member 118 and the single slot being opposed to the single slot of the first retaining member 118.

Each slot 119, 129 has an inner surface shaped to correspond with an outer surface of a surgical clip such that upon applying a force on the surgical clip in a direction towards the inner surface of the slot, the surgical clip is frictionally retained in the slot.

The adaptor of claim 16, wherein the second body retaining member includes at least one second slot member coupled to the inner surface of the second body, the second slot member having a slot extending inwardly into a body of the second slot member.

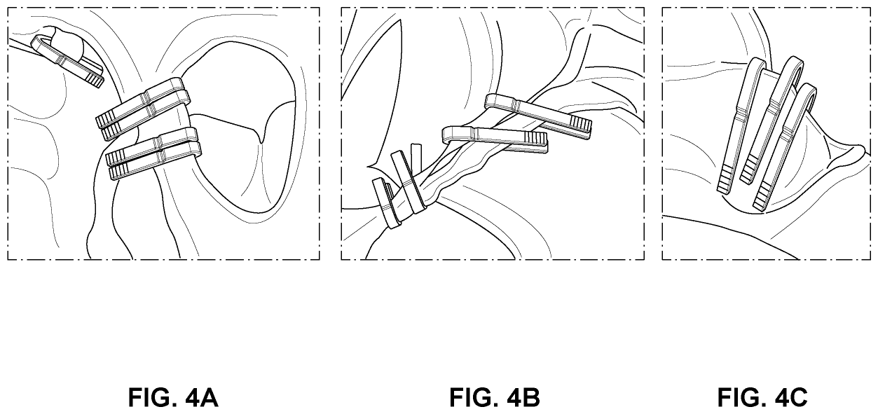

Turning to FIG. 4A, illustrated therein is an image of four clips that were fired using a simultaneous multiclip firing adapter for surgical ligation clip drivers according to at least one embodiment described herein. As is shown therein, the four clips were applied as two pairs of two clips. The two pairs of clips are spaced apart by a distance greater than a distance between each clip of one of the pairs of clips. The four clips are parallel with each other.

FIG. 4B is an image of four clips that were individually placed using a conventional single clip driver. The four clips of FIG. 4B are not aligned with each other and are not parallel with each other.

FIG. 4C is an image of three clips that were individually placed using a conventional single clip driver. The three clips of FIG. 4C are not aligned with each other and are not parallel with each other.

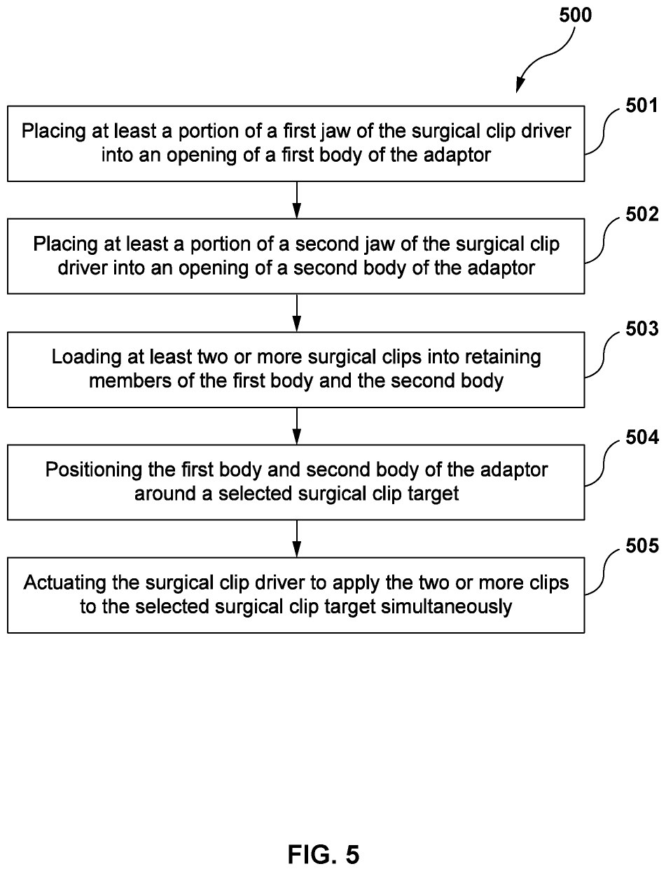

In at least one embodiment, a method 500 of simultaneously applying two or more surgical clips is described herein. FIG. 5 shows a method of simultaneously applying two or more surgical clips, according to at least one embodiment described herein. The method 500 of simultaneously applying two or more surgical clips may be performed with a clip driver and one of the adapters described herein.

At a first step 501, the method 500 includes placing at least a portion of a first jaw of the surgical clip driver into an opening of a first body of the adaptor.

At a second step 502, the method 500 includes placing at least a portion of a second jaw of the surgical clip driver into an opening of a second body of the adaptor.

At a third step 503, the method 500 includes loading at least two or more surgical clips into retaining members of the first body and the second body.

At a fourth step 504, the method 500 includes positioning the first body and second body of the adaptor around a selected target clipping tissue.

At a fifth step 505, the method 500 includes actuating the surgical clip driver to simultaneously apply the two or more clips to the selected target clipping tissue.

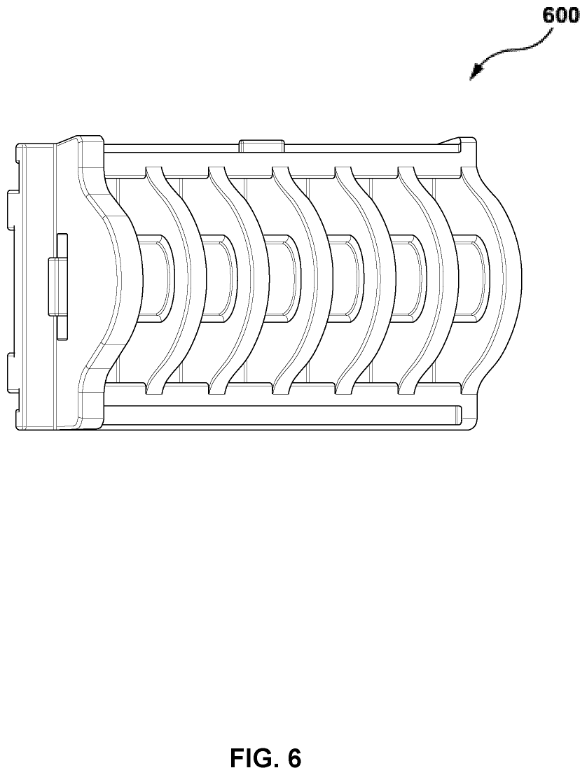

Surgical clip drivers need to be loaded with clips before a surgeon can use the clip driver to apply the clip on to an anatomic structure, such as a vessel or other target tissue. The adaptors described herein need to be simultaneously loaded with two or more clips before a clip driver with an adaptor can be used to simultaneously apply the two or more clips on the anatomic structure. Conventional cartridges generally do not offer the ability to simultaneously load more than one clip onto a surgical clip driver. For example, FIG. 6 shows a perspective view of a conventional cartridge for retaining surgical clips.

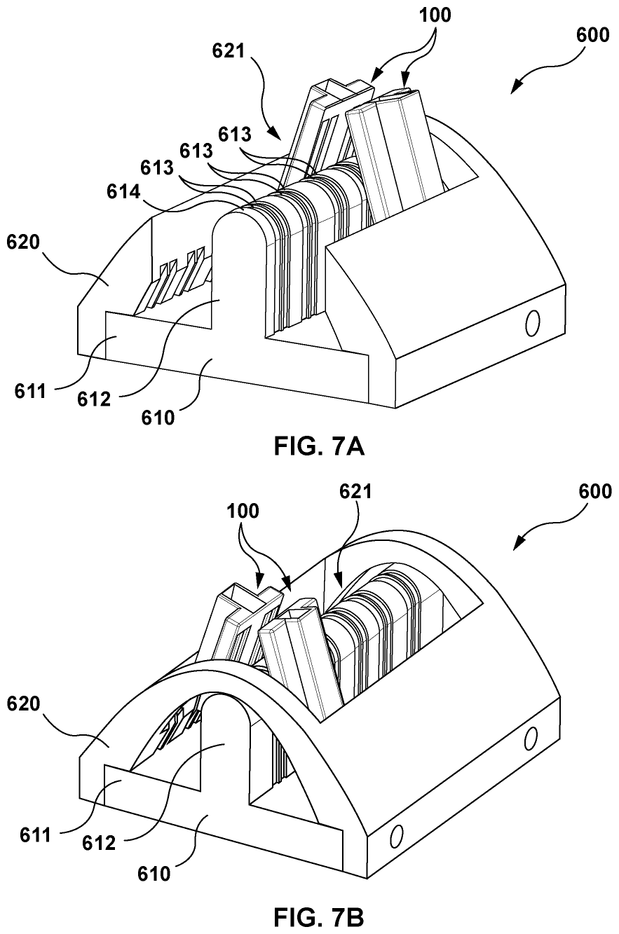

Turning now to FIGS. 7A and 7B, illustrated therein is clip cartridge 600 according to at least one embodiment. Clip cartridge 600 includes a clip support 610 and a cover 620. Clip support 610 includes a base 611 to support a hedge 612 extending upwardly therefrom. Hedge 612 support the two or more clips and includes separators 613 on an outer surface 614 of the hedge 612. In at least one embodiment, separators 613 may be flanges that extend outwardly from hedge 612 and inhibit clips supported by hedge 612 from sliding along a longitudinal axis of hedge 612. Specifically, one separator 613 is positioned on either side of each clip of the cartridge 600 to stable the clips on the hedge 611 a required distance for each other so the clips can be received into the adaptor 100 when the adaptor 100 portion of which is shown in FIGS. 7A and 7B and identified with reference number 100. The distance between the clips on hedge 611 is about the same as the distance between the slots of each adaptor 100, such as distances AA between pairs of slots and distance DD between each slot of each pair of slots, as described above.

Cover 620 is stationed on the base 611 of clip support 610 and includes an opening 621 that is sized and shaped to expose the clips on clip support 610 and receive the adaptor 100 therein. Opening 621 provides for the adaptor 100, when attached to a clip driver, to pass down therethrough and retrieve the clips from clip support 610. Cover 620 may also inhibit the clips from moving in anterior-posterior direction along clip support 610.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. An adaptor for a surgical clip driver for applying two or more surgical clips, the adaptor comprising:
a first body having a first end, a second end, an inner surface extending between the first end and the second end and an outer surface opposed to the inner surface and extending between the first end and the second end, the first end having an opening extending inwardly into the first body, the opening being configured to receive a portion of a first jaw of the surgical clip driver;
a first body retaining member configured to retain a first portion of each of the two or more surgical clips;
a second body having a first end, a second end, an inner surface extending between the first end and the second end and an outer surface opposed to the inner surface and extending between the first end and the second end, the first end having an opening extending inwardly into the second body, the opening being configured to receive a portion of a second jaw of the surgical clip driver; and
a second body retaining member configured to retain a second portion of each of the two or more surgical clips;
wherein, when the first body and the second body are received on the surgical clip driver, the first body retaining member and the second body retaining member face each other and are aligned with each other to receive and retain each of the two or more surgical clips.

2. The adaptor of claim 1, wherein the first body retaining member is integral with the first body and includes two or more slots extending inwardly into the first body and the second body retaining member is integral with the second body and includes two or more slots extending inwardly into the second body.

3. The adaptor of claim 2, wherein the two or more slots of the first body are parallel with each other and extend between the first end of the first body and the second end of the first body and the two or more slots of the second body are parallel with each other and extend between the first end of the second body and the second end of the second body.

4. The adaptor of claim 3, wherein the two or more slots of the first body include two pairs of slots and the two or more slots of the second body include two pairs of slots.

5. The adaptor of claim 4, wherein each the two pairs of slots are spaced apart from each other by a distance that is greater than a distance between the slots of each pair of slots.

6. The adaptor of claim 3, wherein the two or more slots of the first body include three slots.

7. The adaptor of claim 6, wherein the three slots include one pair of slots spaced apart from a single slot.

8. The adaptor of claim 7, wherein the pair of slots is spaced apart from the single slot by a distance that is greater than a distance between the slots of the pair of slots.

9. The adaptor of claim 2, wherein each slot has an inner surface shaped to correspond with an outer surface of a surgical clip such that upon applying a force on the surgical clip in a direction towards the inner surface of the slot, the surgical clip is frictionally retained in the slot.

10. The adaptor of claim 1, wherein the first body retaining member includes at least one first slot member coupled to the inner surface of the first body, the first slot member having a slot extending inwardly into a body of the first slot member.

11. The adaptor of claim 10, wherein the first body retaining member includes three first slot members.

12. The adaptor of claim 11, wherein the three first slot members include one pair of first slot members spaced apart from a single first slot member.

13. The adaptor of claim 12, wherein the pair of first slot members is spaced apart from the single first slot member by a distance that is greater than a distance between the slots of the pair of first slot members.

14. The adaptor of claim 10, wherein the second body retaining member includes at least one second slot member coupled to the inner surface of the second body, the second slot member having a slot extending inwardly into a body of the second slot member.

15. The adaptor of claim 14, wherein the second body retaining member includes three second slot members.

16. The adaptor of claim 15, wherein the three second slot members include one pair of second slot members spaced apart from a single second slot member.

17. The adaptor of claim 16, wherein the second body retaining member includes at least one second slot member coupled to the inner surface of the second body, the second slot member having a slot extending inwardly into a body of the second slot member.

18. The adaptor of claim 17, wherein the pair of second slot members is spaced apart from the single second slot member by a distance that is greater than a distance between the slots of the pair of second slot members.

19. A method of simultaneously applying two or more surgical clips with a surgical clip driver having an adaptor, the method comprising:
placing at least a portion of a first jaw of the surgical clip driver into an opening of a first body of the adaptor;
placing at least a portion of a second jaw of the surgical clip driver into an opening of a second body of the adaptor;
loading at least two or more surgical clips into retaining members of the first body and the second body;
positioning the first body and second body of the adaptor around a selected target clipping tissue; and
actuating the surgical clip driver to simultaneously apply the two or more clips to the selected target clipping tissue.

20. A cartridge for supporting two or more surgical clips, the cartridge comprising:
a clip support configured to support the two or more surgical clips, the clip support including a hedge extending upwardly from a base to support the clips in an upside down orientation such that a rear portion of each clip is upwardly exposed, the hedge having a separator positioned on either side of each clip to stable the clips from each other when the clip support is loaded with two or more clips; and
a cover having an opening sized and shaped to provide for a clip driver to retrieve the two or more clips from the clip support through the opening.

21. (canceled)

22. (canceled)
